(19) United States
(12) Patent Application Publication
(10) Pub. No.: US 2025/0177939 A1
(43) Pub. Date: Jun. 5, 2025

(54) METHOD FOR CONTROLLING AN AMMONIA OR METHANOL CONVERTER

(71) Applicant: CASALE SA, Lugano (CH)

(72) Inventors: Giovanni GENOVA, Como (IT); Sergio PANZA, Como (IT)

(21) Appl. No.: 18/862,080

(22) PCT Filed: May 10, 2023
(86) PCT No.: PCT/EP2023/062400

§ 371 Date: Oct. 31, 2024

(30) Foreign Application Priority Data
May 12, 2022 (EP) 22172957.7

(51) Int. Cl. B01J 8/00; B01J 8/02; C01C 1/04; C07C 29/152

(52) U.S. Cl. CPC B01J 8/001; B01J 8/0278; B01J 8/0285; C01C 1/0417; C01C 1/0482; C07C 29/152; B01J 2208/00407; B01J 2208/00637

(57) ABSTRACT

Method for controlling an ammonia synthesis converter or a methanol synthesis converter during intermittent availability of a renewable power-dependent hydrogen feed, wherein under a limited or no availability of power the converter effluent is recycled back to the inlet of said converter in a loop, and heated to keep said converter in a hot stand-by mode wherein the temperature in the reaction space remains within a target range.

### Field of application

The invention is in the field of chemical plants. In particular, the invention relates to a method for operating an ammonia or a methanol synthesis converter during intermittent availability of renewable energy sources.

### PRIOR ART

The industrial production of ammonia and methanol depends on a hydrogen- containing make-up gas which is commonly produced by reforming a hydrocarbon, such as natural gas.

A common feature of ammonia plants and methanol plants is that the make-up gas is reacted in a so-called synthesis loop. The main items of the synthesis loop include a catalytic converter where the make-up gas is reacted to produce a reaction effluent, a product cooler where the hot reaction effluent is cooled, a separator where a liquid phase containing the product (ammonia or methanol) is removed, a recirculation line where at least a portion of a gaseous phase withdrawn from the separator is recycled back to the converter via a circulator.

The make-up gas is produced in a front-end which comprises the reforming equipment, such as a fired primary reformer and a secondary reformer, and further equipment for processing and purification of the syngas, such as shift conversion and removal of CO₂. The so obtained make-up gas is generally compressed to synthesis pressure in a main compressor. The above mentioned circulator of the synthesis loop may be integrated in the main compressor, i.e. the main compressor and the circulator share the same shaft.

The converter is basically a catalytic reactor with a reaction space (or reactive zone) including one or more catalytic beds, possibly with heat exchangers immersed in the catalytic beds or adiabatic catalytic beds with inter-bed heat exchangers. In normal operation, the make-up gas feed is pre-heated to a suitable reaction temperature by the heat of the exothermic reaction, e.g. by flushing the pressure vessel of the converter and/or in a gas-gas heat exchanger where heat is transferred from the hot reaction effluent to the gas feed. The converter generally comprises a start-up heat exchanger which is used to heat the make-up gas during start-up, when the heat of reaction is not available or insufficient for a proper preheating of the feed.

A reforming-based ammonia or methanol plant may have considerable emission of carbon dioxide into atmosphere, particularly due to fuel-fired primary reformer. There is a growing interest in finding a more environmentally friendly process for production of the required hydrogen feed. In this respect, a very interesting solution is offered by techniques for the production of hydrogen from renewable energy such as electrolysis of water. For example electrolysis of water may be powered with solar energy or wind energy leading to a virtually CO₂-free production of hydrogen (green hydrogen).

However a renewable energy-dependent hydrogen feed is subject to fluctuation. Renewable energy sources are commonly denoted VRE (variable renewable energy sources) due to their intrinsic variability. Soler energy for example is subject to day/night cycles and weather conditions; wind energy typically has fluctuations with higher frequency.

A problem with ammonia or methanol plants fed entirely or partially with such green hydrogen is that the synthesis loop is conventionally designed to run at or close to maximum capacity and is generally not flexible to operate at a partial load. This is particularly true for ammonia converters and methanol converters which are generally not designed to run at a low partial load. The loop and the converter may need 12 to 24 hours startup to reach the nominal synthesis pressure and temperature, so it is not economically acceptable to shut the plant down when the energy source is unavailable.

A shutdown and subsequent startup of the plant should be avoided because not only does it lead to economic losses but further, it may induce a fatigue stress of the converter due to temperature and/or pressure cycles. Furthermore, a reduction in the operating temperature and/or pressure in the converter may lead to condensation of one or more products over the catalyst inducing degradation of the catalyst.

A producer may use energy imported from a grid, when available, to compensate for fluctuations of the renewable energy. However, the grid energy is generally expensive, so this solution is not economically attractive.

Certain solutions that have been proposed in the art rely on the principle of storing of hydrogen and/or heat to deal when the renewable power is largely available, for subsequent use when less or no power is available. These solutions have the drawback of requiring expensive storage means.

EP 3957772 A for example teaches to store hydrogen (H₂) in a buffer tank to feed the ammonia converter during shortage of renewable power. The necessary hydrogen tank is expensive and poses safety concerns in case of leaks. Furthermore, to reduce the size of the storage tank, H₂ must be stored under pressure and for this reason a dedicated compressor is required, which is an additional cost and consumes power.

WO 2021/060985 A1 discloses a solution wherein a heating medium, e.g. molten salt, is stored in a dedicated tank and is used to supply heat to the ammonia converter during limited availability of renewable power so to keep the converter at a suitable temperature for the synthesis. This solution requires the addition of an expensive tank which needs to be large and suitably insulated to store heat.

### SUMMARY OF THE INVENTION

The invention aims to overcome the above drawbacks of the prior art. The invention addresses the problem of how to control an ammonia synthesis loop during shortage of a renewable power-dependent hydrogen feed, to ensure that the loop is able to quickly restart the production of ammonia when the hydrogen feed is restored, avoiding a shutdown of the loop and avoiding the use of expensive storage/buffer equipment. The same problem is addressed for a methanol synthesis loop.

The problem is solved with a method according to claim 1.

When the flow rate of the renewable energy-dependent hydrogen feed is below a threshold value, the method includes recycling at least a portion of the converter effluent back to the inlet of said converter, after passing through the separator, and heating the recycled converter effluent so to keep said converter in a stand- by mode wherein the temperature in the reaction space is within a target range.

The method of the invention maintains the synthesis loop in a hot stand-by mode wherein the reaction space is kept pressurized at a suitable temperature for a quick restart as soon as the renewable power and hydrogen feed are back.

The invention is based on the finding that such hot stand-by mode may be maintained for relatively long periods at the expense of a limited amount of energy.

In a very interesting embodiment of the invention, the heating of the recycled effluent is performed by the start-up heater of the converter. Hence the invention provides an innovative use of the start-up heater, which is exploited not for a start-up transitory, rather to keep the converter in a stand-by mode. The heat input provided by the start-up heater is ingeniously used to compensate for heat loss, or heat possibly removed by a cooling medium that cannot be completely stopped in hot stand-by mode, and to maintain the loop, particularly the reaction space of the converter, in a hot and pressurized condition ready for quick restart of the production, e.g. in just one hour or even less.

For example in an ammonia plant fed with hydrogen from electrolysis of water, a suitable hot stand-by mode may be maintained with a power as low as about 2% or 1% or about 0.5% of the power required for operation at nominal capacity (nominal output of ammonia).

In a preferred embodiment, during the hot stand-by of the invention, the reaction space is maintained below a minimum reaction temperature so that no or negligible synthesis of ammonia or methanol occurs. Accordingly, there is no substantial removal of product from the loop and no introduction of fresh gas into the loop. The loop is maintained in a condition where it is substantially close to mass transfer. The pressure and heat remain within the loop apart from heat losses, or heat removed in the process, which are compensated by the startup heater. To this purpose, the startup heater may be suitably controlled, preferably with an on/off control and/or adjusting its thermal power. The pressure loss of the items and piping is compensated by the compressor or by the circulator.

In the preferred embodiments, the startup heater is an electric heater, however a different embodiment such as fuel-fired heater or heat exchanger may be used.

In the stand-by mode of the invention, the flow rate which circulates in the loop may be according to preferred embodiments not greater than 50% or not greater than 25% or not greater than 10% of the flow rate at nominal capacity.

### DETAILED DESCRIPTION OF THE INVENTION

The invention is further described with reference to an ammonia plant.

The method of the invention is applicable to a plant wherein hydrogen production is at least in part powered by one or more renewable energy sources. The renewable energy sources may include at least one of the following: solar, wind, hydro, geothermal and biomass.

In the stand-by mode the temperature in the reaction space is preferably below an activation temperature of the catalyst. Particularly in case of ammonia synthesis said temperature is preferably not greater than 330° C., preferably not greater than 300° C., more preferably not greater than 260° C. In all the above cases the minimum temperature of the reaction space may be set for example at 150° C. or 200° C. A temperature close to the lower limit of the above range, such as 150° C. to 160° C. or around 150° C., is preferred for the methanol synthesis.

The method of the invention involves keeping the converter and the loop in a stand-by mode when the renewable power available for the production of hydrogen is below a threshold value. The method may be implemented when said power remains below the threshold value for a given time, for example for one hour or more.

Said threshold value may correspond to a capacity, in terms of hydrogen feed that can be produced from the renewable power, which is not greater than 50% or not greater than 25% or not greater than 10% of a nominal hydrogen feed corresponding to a nominal output of the loop, i.e. a nominal amount of ammonia or methanol withdrawn from the loop. The renewable power-dependent hydrogen feed may be the sole hydrogen feed of the loop, which is a preferred embodiment, or may constitute a portion of the hydrogen feed.

Said renewable power-dependent hydrogen feed may be produced with renewable electric power, preferably by water electrolysis.

Said threshold value of the hydrogen feed may corresponds to said renewable electric power being below 50% or below 25% or below 10% of a nominal power corresponding to a nominal output of product.

In a preferred embodiment, the temperature of the reaction space is dynamically controlled by controlling a thermal power transferred to the recycled converter effluent. Particularly preferably, the recycled converter effluent is heated in an electrically powered start-up heater of said converter and the dynamic control of the thermal power transferred to the recycled effluent may include on/off control of the startup heater and/or adjusting the thermal power of the same.

The temperature in the reaction space may be controlled by a suitable control that dynamically regulates the thermal power transferred to the recirculating gas, for example by activating or deactivating the startup heater and/or by adjusting the electrical power output of said startup heater.

Said control unit may be operatively connected to one or more temperature sensors, such as thermocouples, that are configured to sense the temperature at one or more locations in said reaction space. Based on the temperature detected by the sensors, the control unit provides an output signal to the start-up heater.

In a preferred embodiment the standby condition is maintained by the startup heater and circulation of the recycled converter effluent absorbing collectively no more than 2.0% of the electric power required for nominal operation of the plant.

Still further preferred features of the invention are as follows.

The loop typically comprises a synthesis converter, a separator, a circulator and a startup heater of the converter. The startup heater may be fitted internally in the converter or may be a separate item.

The method may comprise recycling at least a portion of the converter gaseous effluent back to the inlet of said converter after passing through the separator and through the startup heater. The recycling can be carried out in a loop that includes at least said circulator, said startup heater, said catalytic converter and said separator.

In the stand-by mode, the reaction space is maintained within a target range of temperature. Said range may be below a minimum reaction temperature so that no or negligible synthesis of said product occurs in the reaction space during the stand-by. Said minimum temperature can be equal to or close to the activation temperature of the catalyst used for the synthesis.

According to an embodiment, no ammonia or methanol are synthesised in the converter, consequently a gaseous flow is continuously recycled in the loop and no transfer of mass outside the loop is carried out. According to an alternative embodiment, a supply of fresh makeup gas and a withdrawal of ammonia or methanol from the loop are periodically carried out to keep the pressure within the loop itself constant.

The loop may further include a condenser that is arranged downstream of said catalytic converter and upstream of said separator.

The startup heater can be arranged upstream of the converter. According to a preferred embodiment, the startup heater is part of the converter and is arranged upstream of the reactive zone, for example above a catalytic bed.

According to a less preferred embodiment, the startup heater may be a fired heater or a shell and tube heat exchanger.

The plant generally includes a makeup gas compressor configured to raise the makeup gas to synthesis pressure.

In some embodiments the circulator of the loop operates independently from the makeup gas compressor. In certain embodiments the circulator and the compressor are part of a single geared machine.

According to an interesting application, the recycling of the gas back to the circulator is made through a dedicated recycle line. In particular, a recycling line can be arranged to take the gaseous flow downstream of the converter and to re-introduce the gas at a suitable location upstream of said circulator.

A dedicated recycle line may provide a shorter recycle path and reduce the pressure drops and loss of heat, e.g. the gaseous effluent may not need to pass through the condenser and separator but it can be recycled back directly to the suction section of the circulator.

Said recycle line may be provided with a heat exchanger to cool down the gas. Preferably, the gas is cooled to a temperature of about 50 to 60° C. prior to be conveyed to the circulator.

Preferably, said plant does not comprise a hydrogen buffer tank and/or a utility tank used to store a heating medium. Hence in a preferred embodiment the method of the invention does not include a storage of hydrogen and/or storage of heat. In some embodiments a storage may be provided, however the invention is still advantageous in that the storage size and cost are greatly reduced compared to solutions which entirely rely on storage to cope with fluctuation of renewable power.

In a very interesting application, the method of the present invention cooperates with a method for controlling a loop at partial load according to the disclosure of WO 2021/089276. Said method includes: separating a gas stream from the converter feed line, at a point upstream of the converter, to form a bypass stream and reintroducing said bypass stream at the suction side of the circulator or into the synthesis loop at a point downstream of said separation section.

Accordingly, when the renewable power fluctuates, the method of the present invention may include the following.
a) If the renewable power remains above a predetermined threshold, the loop is controlled at a partial load by separating a gas stream from the converter feed line, at a point upstream of the converter, to form a bypass stream, and reintroducing said bypass stream at the suction side of the circulator or into the synthesis loop at a point downstream of said separation section, and possibly according to further details disclosed in WO 2021/089276;
b) If the renewable power falls below a minimum value, the loop moves to the hot stand-by mode of the present invention.

Under the condition a) the loop continues to produce ammonia (or methanol) although at a reduced capacity, which may be as low as 10% of the nominal capacity. Under the condition b), preferably, the synthesis is interrupted or almost interrupted because the loop is kept below the catalyst activation temperature.

Preferably, under the condition b) of hot stand-by, there is no product or substantially no product withdrawn from the loop. The term substantially no product may denote that the amount of product removed from the loop in the stand-by mode is less than 5%, preferably less than 1%, of the amount removed during normal operation.

A plant may be controlled to switch from a) to b) at a given fraction of the nominal load. In mode a), the loop may follow the fluctuation until the load of the converter is equal to or greater than said fraction of the nominal load; if the hydrogen feed and the load tend to fall even below, the loop enters the hot stand-by mode b) until the power returns above said fraction. The control system may include that switch from a) to b) is performed at a first fraction of the nominal load and switch from b) to a) is performed at a second fraction of the nominal load. The second fraction may be equal to or different from the first fraction. The second fraction may be greater than the first fraction to provide a more stable operation. The first load fraction, at which the system enters the hot stand-by mode b), is preferably in the range 5% to 50%, for example 5%, 10%, 15%, 20%, 25%, 30%, 35%, 40%, 45%. The second load fraction, at which the system returns to normal mode a), may be, for example, any of the above-listed values.

A combination of the variable load control method of WO 2021/089276 and of the hot stand-by control of the present invention is particularly synergistic to optimise the use of a renewable energy source in the ammonia or methanol production.

As a practical example, an ammonia plant rated at 53 MTD (metric tons/day) of ammonia fed with green hydrogen from a solar-powered electrolyzer requires around 21,000 KW for operation at nominal capacity. Assuming to keep the reaction space at a temperature of about 250° C., it has been calculated that a startup heater may require around 85 KW and circulation may require an additional 40 KW. Accordingly, the hot stand-by mode may be maintained with an input of 125 KW equal to about 0.6% of the nominal power.

Still further aspects of the invention are according to the following points:
1) A method for controlling a process for production of ammonia or methanol from a make-up gas containing hydrogen produced from renewable power, wherein said production of ammonia or methanol involves the conversion of said make-up gas in a catalytic reactor, wherein during limited or no availability of said renewable power the reactor is kept in a hot stand-by mode by a continuous loop re-circulation of a gas flow which is heated to keep the reactor under pressure and within a temperature range, said temperature range being below activation temperature of the catalyst so that substantially no ammonia or methanol is produced during the stand-by.
2) A method according to point 1 above wherein said gas flow is heated in a startup heater of the reactor.
3) A method according to points 1 or 2 above wherein the hot stand-by mode is implemented when the renewable power falls below a minimum power corresponding to a minimum acceptable load of the reactor.
4) A method according to any of the above points 1 to 3 wherein no hydrogen storage is performed.
5) A method according to any of the above points 1 to 4 wherein loop circulation in the stand-by mode is maintained by a make-up gas compressor and/or by a circulator of a synthesis loop.

### DESCRIPTION OF THE FIGURES

The invention is further described with reference to the figures wherein:

FIG. 1 is a schematic representation of an ammonia synthesis plant according to an embodiment;

FIG. 2 is a schematic representation of an ammonia synthesis plant according to an alternative embodiment.

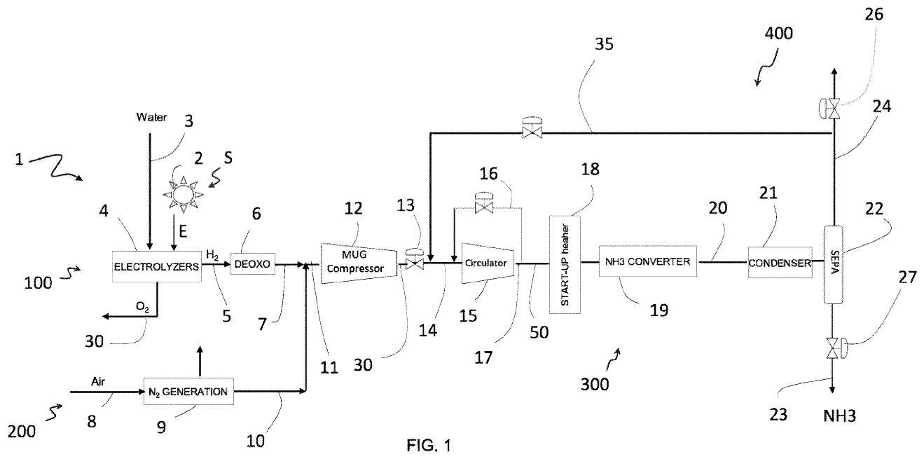

The ammonia plant 1 of FIG. 1 includes: a hydrogen generation section 100 for the generation of a hydrogen stream 7; a nitrogen production section 200 for the production of a nitrogen feed 10; an ammonia synthesis section 300 where said hydrogen stream 7 and said nitrogen feed 10 are reacted to form ammonia product 23.

More in detail, the hydrogen generation section 100 includes a water electrolyzer 4 for the generation of a hydrogen feed 5 from a water stream 3 and an oxygen stream 30. The electrolyzer 2 is powered by electric power E provided by a renewable source 2 which in FIG. 1 is a solar source S. The solar source S may be for example a photovoltaic field.

The hydrogen section 100 further includes a deoxygenation unit 6 configured to remove traces of oxygen from the hydrogen feed 5. Output of the deoxygenation unit is the hydrogen stream 7.

The nitrogen production section 200 includes a nitrogen generation unit 9 for the extraction of nitrogen 10 from an air feed 8. Said nitrogen generation unit 9 can be an air separation unit (ASU) that also produces oxygen or oxygen-enriched air.

The hydrogen stream 7 and the nitrogen feed 10 are mixed together to yield a makeup gas 11 that is delivered to the ammonia synthesis section 300 via a make-up gas compressor 12. Effluent of the make-up gas compressor 12 is a compressed makeup gas 30. A valve 13 is arranged downstream of the makeup gas compressor 12 and upstream of the ammonia synthesis section 300 to regulate the flow of compressed makeup gas delivered to ammonia synthesis section.

The ammonia synthesis section 300 includes an ammonia converter 19 and a circulator 15 equipped with a bypass line 16. The circulator 15 receives the compressed make-up gas 30 effluent of the compressor 12 and it also receives a recycle gas 24 effluent of a separator 22. The recycle gas 24 and the compressed make-up gas 30 can be mixed together to yield a mixed stream 14 before being supplied to the circulator 15. In other embodiments the recycle gas 24 and the compressed make-up gas 30 can be supplied to the circulator 15 as separate streams, or the make-up gas 30 can be directly fed downstream to the circulator 15.

Effluent of the circulator 15 is a reagent gas feed 17 that is conveyed to the ammonia converter 19. The ammonia converter 19 includes one or more reactive zone(s) e.g. one or more catalytic bed(s) and a startup heater 18 that is arranged upstream of the reactive zone(s). In FIG. 1, for simplicity, the startup heater 18 is illustrated as a separate item prior to the ammonia converter 19 but preferably the startup heater 18 is part of the converter 19, e.g. mounted internally in a top part of the pressure vessel of the converter.

The bypass line 16 can be used to recycle a portion of the reagent gas feed 17 of the circulator 15 to keep the operating pressure of the ammonia converter 19 within a pre-established range especially during partial load events as disclosed in WO2021/089276 A1. According to various embodiments, other means such as throttling a suction circulator valve can be provided to keep the loop pressure almost constant.

In the ammonia converter the reagent gas feed 17 is reacted over a suitable catalyst to form a gaseous effluent 20 containing ammonia. The gaseous effluent 20 containing ammonia is then cooled in the condenser 21 and the effluent of the condenser is then conveyed to a separator 22 wherein ammonia 23 is separated from the recycle gas 24. The amount of ammonia 23 withdrawn from the separator 23 is regulated via the valve 27.

At least a portion of the recycle gas 24 is recycled to the suction section of the circulator 15 via line 35, where it is mixed with the compressed make-up gas 30.

The converter 19 is part of a loop 400 including the circulator 15, the startup heater 18 and converter 19, the condenser 21, the separator 22 and the return line 35.

A portion of the recycle gas 24 can be discharged via the valve 26 to avoid the accumulation of inerts in the loop 400.

The method of the invention is now explained with reference to FIG. 1

When the renewable power E is limited or not available the water electrolyzer 4 is not able to provide the hydrogen feed 7. To prevent the complete shutdown of the plant, the gas delivered by the circulator 15 and traversing the converter 19, condenser 21 and separator 22 is continuously recycled via the line 35 to the suction of said circulator 15, i.e. within the loop 400.

The continuously recycled gas is heated by the startup heater 18 which is preferably an electrical heater that is operatively connected to a control system (not shown in the figure) which in turn is connected to a temperature sensing device. The temperature sensing device measures one or more temperature(s) in the reactive zone(s) of the converter 19 and provides a signal to the control unit. When the temperature(s) measured in said reactive zone(s) is/are lower than a threshold value(s) the control unit activates the startup heater and regulates the electrical power output of the heater so to keep the temperature in said reactive zone(s) at a stand-by temperature that is below the activation temperature of the catalyst in the converter 19, preferably between 200° C. and 330° C., more preferably between 200° C. and 260° C.; in case of methanol synloop preferably the set temperature should be close to 150° C.

The loop is kept in a hot stand-by mode where substantially no ammonia is synthesized. The gaseous effluent 20 of the circulator 19 that is continuously recycled in the loop 400 mainly comprises hydrogen and nitrogen. In such stand-by mode, substantially no ammonia is condensed in the condenser 21 and separated in the separator 22.

The loop 400 during the stand-by mode is substantially a closed loop. The valves 13, 26 and 27 accordingly may be closed.

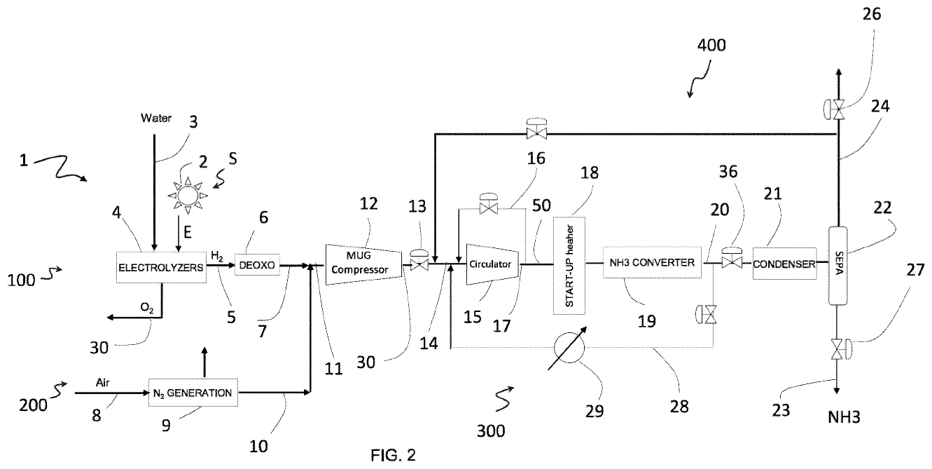

FIG. 2 shows an alternative embodiment of the invention wherein a dedicated recycle line 28 is provided. Said line 28 connects a point downstream the converter 19 but upstream the condenser 21 to the suction side of the circulator 15. In the stand-by condition, the gas is recycled via said line 28 avoiding a passage through the condenser 21 and separator 22.

Preferably, a heat exchanger 29 is provided in the line 28 to cool down the gas. According to the present embodiment, the closed-loop may be obtained by closing the valve 13 arranged after the make-up compressor 12 and valve 36 prior to the condenser.
1-17. (canceled)

18. A method for controlling an ammonia synthesis converter or a methanol synthesis converter during intermittent availability of a renewable power-dependent hydrogen feed, wherein:
said converter includes a reaction space containing a catalyst and configured to react a reagent gas, including said hydrogen feed, to form ammonia or methanol;
wherein, in a condition when the renewable power available for generation of the hydrogen feed is below a threshold value, the method comprising:
recycling at least a portion of the converter effluent back to an inlet of said converter in a loop; and
heating the recycled converter effluent so to keep said converter in a stand-by mode, wherein a temperature in the reaction space is within a target range;
wherein the recycled converter effluent is heated in a startup heater of said converter.

19. The method according to claim 18, wherein said target range of temperature is below a minimum reaction temperature so that no or negligible synthesis of said product occurs in the reaction space during the stand-by mode.

20. The method according to claim 19, wherein, in said stand-by mode, no product or substantially no product is removed from the loop and no fresh reaction gas is introduced in the loop.

21. The method according to claim 18, wherein in the stand-by mode the temperature in the reaction space is in the range 150° C. to 330° C.

22. The method according to claim 18, wherein said threshold value corresponds to a capacity in terms of hydrogen obtainable from the renewable power which is not greater than 50% of a nominal hydrogen feed corresponding to a nominal output capacity of the converter.

23. The method according to claim 18, wherein said hydrogen feed is produced with renewable electric power via water electrolysis.

24. The method according to claim 23, wherein said threshold value corresponds to said renewable electric power being below 50% of a nominal power corresponding to a nominal output of the converter.

25. The method according to claim 23, wherein said renewable electric power is produced by one or more renewable energy sources including solar energy.

26. The method according to claim 18, wherein the temperature of said reaction space, during the stand-by mode, is dynamically controlled by controlling a thermal power transferred to the recycled converter effluent.

27. The method according to claim 18, wherein the startup heater is arranged upstream of the converter, or is part of the converter and is arranged upstream of a reactive zone above a catalytic bed.

28. The method according to claim 18, wherein said startup heater is electrically powered.

29. The method according to claim 28, further comprising on/off controlling of said electric startup heater to control the temperature in the reaction space.

30. The method according to claim 28, wherein the standby mode is maintained by the startup heater and circulation of the recycled converter effluent absorbing no more than 2.0% of the electric power required for nominal operation of the plant.

31. The method according to claim 18, wherein:
the converter is part of a synthesis loop that includes, further to the converter, a circulator, a pre-heater of the converter feed, a condenser downstream the converter, a separator downstream the condenser, a recycle gas line connecting said separator to a point upstream said circulator; and
during said stand-by mode of the converter, the effluent is recycled to the converter via said tail gas line.

32. The method according to claim 18, wherein
the converter is part of a synthesis loop that includes, further to the converter, a circulator, a pre-heater of the converter feed, a condenser downstream the converter, a separator downstream the condenser, a tail gas line connecting said separator to a point upstream said circulator; and
during said stand-by mode of the converter, the effluent is recycled to the converter via a dedicated line connecting a point downstream the converter but upstream the condenser to a point upstream said circulator.

33. The method according to claim 18, wherein the flow rate of gas that cycles the loop during the stand-by mode is not greater than 50% of the total flow rate through the converter at nominal capacity.

34. The method according to claim 18, wherein the method is performed without a buffer storage of hydrogen and without a buffer storage of heat.
